(19) United States
(12) Patent Application Publication
(10) Pub. No.: US 2022/0319052 A1
(43) Pub. Date: Oct. 6, 2022

(54) OCT DEVICE, OCT DATA PROCESSING METHOD, PROGRAM AND RECORDING MEDIUM

(71) Applicant: NIDEK CO., LTD., Gamagori-shi (JP)

(72) Inventors: Hisanari TORII, Gamagori-shi (JP); Kenshiro FUJIU, Gamagori-shi (JP); Ryosuke SHIBA, Gamagori-shi (JP); Yukihiro HIGUCHI, Gamagori-shi (JP); Tetsuya KANO, Gamagori-shi (JP); Junpei NISHIYAMA, Gamagori-shi (JP); Kenji NAKAMURA, Gamagori-shi (JP); Norimasa SATAKE, Gamagori-shi (JP)

(21) Appl. No.: 17/708,635

(22) Filed: Mar. 30, 2022

(30) Foreign Application Priority Data
Mar. 31, 2021 (JP) 2021-061791
May 12, 2021 (JP) PCT/JP2021/018022

(51) Int. Cl. G06T 9/00; G06T 11/00; G06T 7/60; G06T 7/174; G16H 30/40

(52) U.S. Cl. CPC G06T 9/00; G06T 11/008; G06T 7/60; G06T 11/005; G06T 7/174; G16H 30/40; G06T 2207/10101; G06T 2207/30041; G06T 2211/404; G06T 2200/24; G06F 3/0482

(57) ABSTRACT

This invention stores necessary OCT data and reduces unnecessary OCT data. The OCT device includes: an OCT optical system that captures an image of a subject eye in accordance with a measurement action; a data obtaining section that obtains OCT data; a subject data determining section that determines non-reduction-subject data or reducing process section; a reducing process section that reduces a data amount of the reduction subject data or delete the reduction subject data; and a storage executing section that stores the non-reduction-subject data and after-reduction data in a storage section.

This Nonprovisional application claims priority under 35 U.S.C. § 119 on Patent Application No. 2021-061791 filed in Japan on Mar. 31, 2021 and PCT/JP2021/018022 filed in W.I.P.O. on May 12, 2021, the entire contents of which are hereby incorporated by reference.

### TECHNICAL FIELD

The present invention relates to an optical coherence tomography (OCT) device, an OCT data processing method (i.e., a method for processing OCT data), and the like.

### BACKGROUND ART

Conventionally, there have been known OCT devices that obtain OCT data (e.g., a tomographic image) of a retina of a subject in accordance with reflected light of measurement light and reference light. The OCT devices continue to evolve to capture an image at a deeper position with a higher resolution.

Patent Literature 1 discloses the following technique. That is, after three-dimensional OCT data is obtained, a scanning line according to which data is to be extracted is determined. Then, from the three-dimensional OCT data, a two-dimensional tomographic image corresponding to the scanning line thus determined is extracted and displayed on a screen.

### CITATION LIST

### Patent Literature

Patent Literature 1

Japanese Patent Application Publication, Tokukai, No. 2019-150532

### SUMMARY OF INVENTION

### Technical Problem

Recently, the size of data obtained by OCT scanning is increasing, and accordingly the size of data to be stored in a storage section is enormously increased. An aspect of the present invention has an object to provide an OCT device and the like that can store necessary OCT data and reduce unnecessary OCT data.

### Solution to Problem

In order to attain the above object, an OCT device in accordance with an aspect of the present invention includes: an OCT optical system that captures an image of a subject eye of a subject in accordance with a measurement action; a data obtaining section that obtains plural pieces of OCT data in accordance with the measurement action; a subject data determining section that determines, on a per-piece-of-data basis, each of the plural pieces of OCT data as (i) non-reduction-subject data or (ii) reduction subject data whose data amount is to be reduced more than that in a case where the each of the plural pieces of OCT data is determined as the non-reduction-subject data or which is to be deleted; a reducing process section that carries out a data amount reducing process or a deleting process on the reduction subject data determined by the subject data determining section; and a storage executing section that stores, in a storage section, the non-reduction-subject data determined by the subject data determining section and after-reduction data obtained as a result of the data amount reducing process carried out on the reduction subject data by the reducing process section.

In order to attain the above object, a method in accordance with an aspect of the present invention for processing OCT data includes: an image-capturing step of capturing an image of a subject eye of a subject in accordance with a measurement action; a data obtaining step of obtaining plural pieces of OCT data in accordance with the measurement action; a subject data determining step of determining, on a per-piece-of-data basis, each of the plural pieces of OCT data as (i) non-reduction-subject data or (ii) reduction subject data whose data amount is to be reduced more than that in a case where the each of the plural pieces of OCT data is determined as the non-reduction-subject data or which is to be deleted; a subject data confirming step of allowing confirmation of whether or not the determination of the reduction subject data made in the subject data determining step is appropriate; a reducing process step of carrying out a deleting process or a data amount reducing process on the reduction subject data determined in the subject data determining step; and a storage executing step of storing the non-reduction-subject data determined in the subject data determining step and after-reduction data that is obtained as a result of the data amount reducing process carried out on the reduction subject data in the reducing process step.

An OCT device in accordance with each aspect of the present invention may be realized by a computer. In this case, the present invention encompasses (i) a program for causing a computer to operate as each section (software element) in the OCT device to realize the OCT device and (ii) a computer-readable storage medium in which the program is stored.

### Advantageous Effects of Invention

In accordance with an aspect of the present invention, it is possible to provide an OCT device and the like that can store necessary OCT data and reduce unnecessary OCT data.

### BRIEF DESCRIPTION OF DRAWINGS

FIG. 1 is a block diagram illustrating an example of a configuration of a main part of an OCT device in accordance with Embodiment 1 of the present invention.

FIG. 2 is a view illustrating an example of a presenting screen presented by a presenting screen.

FIG. 3 is a view illustrating an example of the presenting screen presented by the presenting screen.

FIG. 4 is a view illustrating an example of the presenting screen presented by the presenting screen.

FIG. 5 is a view illustrating examples of kinds of reports.

FIG. 6 is a view illustrating an example of the presenting screen presented by the presenting screen.

FIG. 7 is a view illustrating an example of the presenting screen presented by the presenting screen.

FIG. 8 is a flowchart illustrating a flow of a process to be executed by the OCT device in accordance with Embodiment 1 of the present invention.

FIG. 9 is a block diagram illustrating an example of a configuration of a main part of an OCT device in accordance with Embodiment 2 of the present invention.

FIG. 10 is a block diagram illustrating an example of a configuration of a main part of an OCT device in accordance with Embodiment 3 of the present invention.

### DESCRIPTION OF EMBODIMENTS

### Embodiment 1

The following description will discuss, in detail, an embodiment of the present invention.

Configuration of OCT Device 1

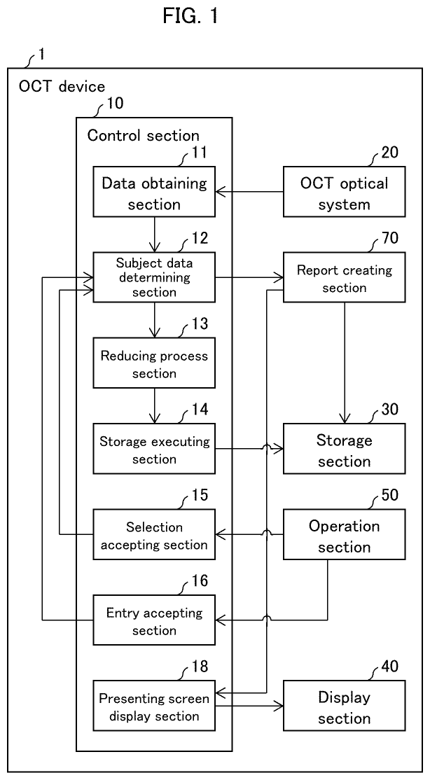

A configuration of an OCT device 1 will be discussed with reference to FIG. 1. FIG. 1 is a block diagram illustrating an example of a configuration of a main part of the OCT device 1 in accordance with Embodiment 1 of the present invention.

The OCT device 1 includes an OCT optical system 20, a control section 10, a storage section 30, and a display section 40. The OCT device 1 may further include an operation section 50 and a report creating section 70.

OCT Optical System 20

The OCT optical system 20 is an optical device that captures an image of a subject eye of a subject in accordance with a preset measurement action. In an example, the OCT optical system 20 includes an OCT light source, a branching optical element, an irradiation optical system, a multiplexing optical element, and a light-receiving element. However, this is not limitative.

The preset measurement action may be, for example, information specifying a scanning pattern along which the OCT optical system 20 is caused scan the subject eye and/or information specifying a scanning position of the subject eye. The measurement action may also include information specifying a scanning pattern and/or a content (a portion of the subject eye) of data to be transmitted to the later-described data obtaining section 11 after image-capturing, for example.

Storage Section 30

The storage section 30 stores therein non-reduction-subject data and after-reduction data (each of which will be described later), for example. The storage section 30 may further store therein a plurality of templates to be combined with OCT data. The storage section 30 may store therein a database of normative eyes that can be used in the templates. The storage section 30 may store therein a report obtained by combining OCT data with a given template.

The non-reduction-subject data may be data that is OCT data whose data amount is not to be reduced. Alternatively, as will be described later, the non-reduction-subject data may be data to be subjected to a data amount reducing process by which data is reduced in an amount smaller than that in a process to be carried out on reduction subject data.

The entity in which the non-reduction-subject data and the after-reduction data are to be stored is not limited to a storage section included in the OCT device 1, such as the one shown in FIG. 1. Alternatively, the non-reduction-subject data and the after-reduction data may be stored in an external storage (e.g., a storage server in a facility) or a cloud storage. The number of entities in which the non-reduction-subject data and the after-reduction data are to be stored may be two or more. In a case where a storage to store data can be selected from a plurality of storages, the data may be determined as non-reduction-subject data or reduction subject data according to which of storages the data is stored in.

Display Section 40

The display section 40 may present a presenting screen designated by the later-described presenting screen display section 18, for example. The display section 40 may be a display of the OCT device 1, for example.

Operation Section 50

The operation section 50 accepts operation carried out by a worker. The operation section 50 may be, for example, a touch panel, a controller, a button, and/or a keyboard. The operation section 50 may output a signal corresponding to the accepted operation to a selection accepting section 15 and/or an entry accepting section 16 (each of which will be described later), for example.

Report Creating Section 70

The report creating section 70 creates a report by combining OCT data with a template (described later). The report creating section 70 may create the report at any of the following timings (i) to (iv): (i) a timing after the selection accepting section 15 selects the template for the report but before a subject data determining section 12 determines non-reduction-subject data or reduction subject data; (ii) a timing after the selection accepting section 15 selects the template for the report and the subject data determining section 12 determines non-reduction-subject data or reduction subject data; (iii) a timing after a reducing process section 13 carries out a reducing process; and (iv) a timing after a storage executing section 14 stores the reduction subject data or the non-reduction-subject data in the storage section 30.

Control Section 10

The control section 10 obtains OCT data from the OCT optical system 20. The control section 10 includes the data obtaining section 11, the subject data determining section 12, the reducing process section 13, the storage executing section 14, the selection accepting section 15, the entry accepting section 16, and the presenting screen display section 18.

Data Obtaining Section 11

The data obtaining section 11 obtains plural pieces of OCT data in accordance with a preset measurement action. The plural pieces of OCT data thus obtained may be plural pieces of two-dimensional OCT data or three-dimensional OCT data including plural pieces of two-dimensional OCT data. The “two-dimensional OCT data” refers to OCT data indicating two-dimensional information extending in a direction intersecting an optical axis (not illustrated) of the OCT optical system 20. The “three-dimensional OCT data” refers to data indicating three-dimensional information extending in a direction intersecting the optical axis and a direction along the optical axis. The OCT data may include data having been subjected to an analysis process.

The OCT data may include information of at least one of a macular portion, an optic papilla portion, and vasa sanguinea retinae of the subject eye. In a case where the OCT data is image data, the image data may include at least one of the macular portion, the optic papilla portion, and the vasa sanguinea retinae of the subject eye.

The OCT data may include, in addition to the fundus, at least one of portions of the eye such as cornea, anterior chamber angle, sclera, and or a serrata.

The plural pieces of OCT may be obtained in a single examination, since the OCT device 1 requires plural pieces of OCT data to analyze the condition of the eye. The subject data determining section 12 may determine, as non-reduction-subject data or reduction subject data, each of the plural pieces of OCT data obtained in the single examination, the determination being made on a per-piece-of-data basis.

The single examination may include an examination on only one of the eyes of a single subject or an examination on both of the eyes of the single subject. The single examination may be examinations carried out on a single subject on a single day.

Subject Data Determining Section 12

The subject data determining section 12 determines whether to set the obtained OCT data as non-reduction-subject data which is to be stored without being subjected to data amount reduction. The subject data determining section 12 also determines the obtained OCT data as (i) reduction subject data whose data amount is to be reduced more than that in a case where the obtained OCT data is determined as non-reduction-subject data or (ii) deletion subject data which is to be deleted. Alternatively, the subject data determining section 12 may determine the obtained OCT data as (i) non-reduction-subject data which is to be stored without being subjected to data amount reduction or (ii) reduction subject data whose data amount is to be reduced more than that in a case where the obtained OCT data is determined as non-reduction-subject data. Further alternatively, the subject data determining section 12 may determine the obtained OCT data as (i) non-reduction-subject data or (ii) deletion subject data which is to be deleted. Still further alternatively, the subject data determining section 12 may determine the obtained OCT data as non-reduction-subject data, reduction subject data, or deletion subject data. Note that the reduction subject data and the deletion subject data may collectively be called “reduction subject data”. The determination of the non-reduction-subject data, the reduction subject data, or the deletion subject data may be carried out for each measurement action or for each subject, for example. Carrying out the determination for each measurement action means that, every time the OCT optical system 20 carries out a single measurement action, the OCT data is determined as non-reduction-subject data, reduction subject data, or deletion subject data, for example. A subject data determining method to be carried out by the subject data determining section 12 will be described later. The measurement action is, for example, image-capturing of a subject eye carried out by the OCT optical system 20. The measurement action may be carried out in accordance with a preset scanning pattern. A single measurement action may include a plurality of scanning patterns.

The subject data determining section 12 determines non-reduction-subject data, reduction subject data, or deletion subject data for each measurement action or for each subject. With this, it is possible to deal with data for each measurement action or for each subject, whereas this is impossible in a case where data to be stored and data not to be stored are collectively determined. This can reduce the possibility of occurrence of a situation in which necessary OCT data is not stored and/or a situation in which unnecessary OCT data is stored.

Reducing Process Section 13

The reducing process section 13 carries out a deleting process on the deletion subject data determined by the subject data determining section 12. The deleting process herein carries out irreversible deletion.

The reducing process section 13 carries out a data amount reducing process on the reduction subject data determined by the subject data determining section 12. Examples of the data amount reducing process encompass a process of (reversibly or irreversibly) compressing OCT data, a process of reducing a resolution of OCT data, a process of deleting OCT data of a given pattern, and a process of deleting OCT data of a given portion. The process of (reversibly or irreversibly) compressing the OCT data may be a process of deleting parts other than a feature necessary for analysis, for example. Specifically, this process may be a process of deleting, in a two-dimensional tomographic image, a line(s) other than a line corresponding to a layer indicating a feature of the subject eye. Note that the lines are obtained as a result of an analysis process (segmentation process). For example, the process of reducing the resolution of the OCT data may be carried out in the following case. That is, a user wishes to keep OCT data so as to use it as a thumbnail, although he/she would not use the OCT data directly for diagnosis. The process of deleting the OCT data of the given pattern may be a process of deleting, in a case where a measurement action including a plurality of times of image-capturing is selected prior to execution of image-capturing, OCT data obtained by image-capturing determined as unnecessary after execution of the image-capturing, for example. The process of the deleting OCT data of the given portion may be a process of deleting, in a front image, an area other than the given area, for example. Specifically, this process may be a process of deleting an area other than an abnormal portion (described later).

Storage Executing Section 14

The storage executing section 14 stores, in the storage section 30, the non-reduction-subject data determined by the subject data determining section 12 and the after-reduction data obtained as a result of the process carried out by the reducing process section 13.

The storage executing section 14, which stores the non-reduction-subject data and the after-reduction data for each measurement action and/or for each subject, may also store, in the storage section 30, OCT data (given OCT data) obtained at a preset scanning position in the subject eye of the subject. The preset scanning position may be (i) coordinates indicating a scanning position of the subject eye or (ii) a certain position in the subject eye. With this, it is possible to store, in the storage section 30, the OCT data obtained at the preset scanning position, independently of a preset measurement action and/or a subject.

Presenting Screen Display Section 18

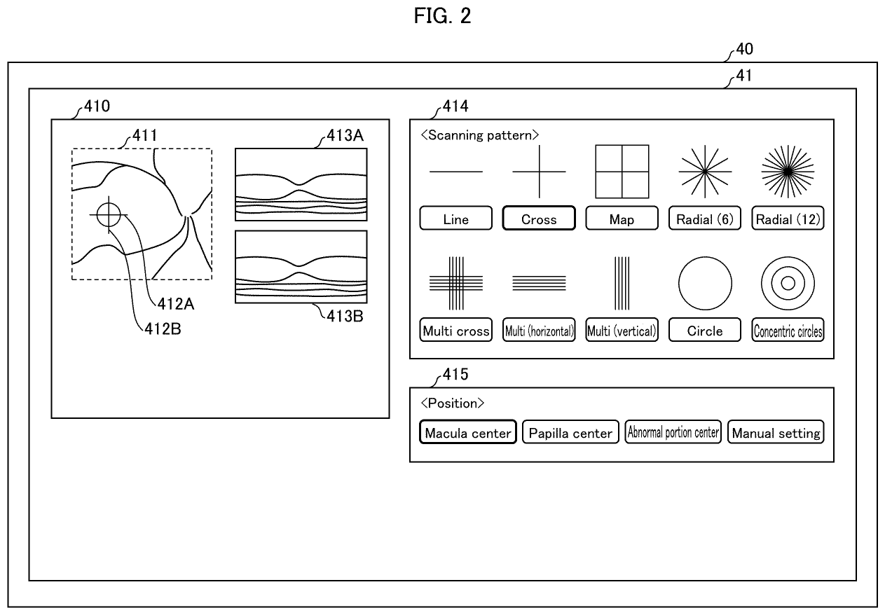

Before image-capturing, the presenting screen display section 18 may display, on the display section 40, a presenting screen that allows selection of a measurement action in advance. FIG. 2 shows an example of a presenting screen 41 that allows selection of a scanning pattern presented by the display section 40. As shown in FIG. 2, the presenting screen 41 presents, for example, a subject eye display area 410, a scanning pattern selection area 414, and a position selection area 415. The subject eye display area 410 indicates a real-time image of a given position of the subject eye captured by the OCT optical system 20. In an example, the subject eye display area 410 indicates a two-dimensional front image 411 and two-dimensional tomographic images 413A and 413B. In an example, the two-dimensional tomographic image 413A is a two-dimensional tomographic image taken in a cross section intersecting a line 412A, whereas the two-dimensional tomographic image 413B is a two-dimensional tomographic image taken in a cross section intersecting a line 412B. The scanning pattern selection area 414 shows options of scanning patterns for image-capturing carried out by the OCT optical system 20. The position selection area 415 shows options of positions in the subject eye where image-capturing is to be carried out by the OCT optical system 20.

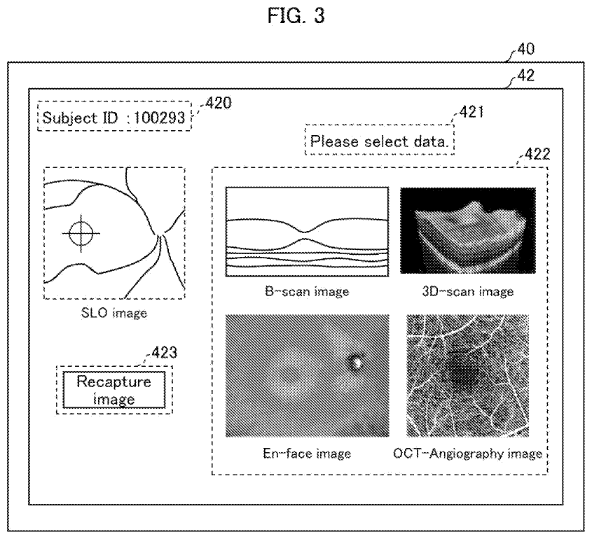

The presenting screen display section 18 may display, on the display section 40, a presenting screen that allows determination of the OCT data as non-reduction-subject data, reduction subject data, or deletion subject data. FIG. 3 illustrates an example of the OCT data presented by the display section 40 and a presenting screen 42 that allows selection of deletion subject data. As shown in FIG. 3, the presenting screen 42 presents, for example, an identification information area 420 that displays a subject ID, a text display area 421 that displays a text, and an OCT data selection area 422 that allows selection of non-reduction-subject data. The text display area 421 may present a message that urges selection of data, e.g., a message “Please select data to delete.” The OCT data selection area 422 may indicate plural pieces of OCT data of given image types. The given image types may be, e.g., formats of a B-scan image, a 3D-scan image, an En-face image, and an OCT-Angiography image. The B-scan image is a two-dimensional tomographic image. The 3D-scan image is a three-dimensional fundus image made of a plurality of two-dimensional tomographic images. The En-face image is a front plane image obtained through reconfiguration of a plurality of two-dimensional tomographic images. The OCT-Angiography image is an image showing the eye with a blood flow. The given image types may include an analysis result, which is a result of analysis of OCT data. Examples of the analysis result encompass (i) a thickness map generated by thickness calculation from a two-dimensional tomographic image and (ii) an image indicating a thickness distribution generated by thickness calculation for a certain part corresponding to a scanning line on the basis of a result of given scanning. The OCT data selection area 422 may be presented together with a front fundus image that indicates (i) a subject portion of the subject eye and (ii) a scanning pattern. The front fundus image may be, for example, a confocal scanning laser ophthalmoscope (SLO) image, an fundus camera image, or an En-face image.

FIG. 3 shows the aspect that allows selection of only the deletion subject data. After the deletion subject data is selected, a presenting screen that allows selection of reduction subject data or non-reduction-subject data may be presented. For example, in a case where selection of non-reduction-subject data is to be carried out, the text display area 421 may present a message that urges selection of data, e.g., a message “Please select data to store.” Meanwhile, in a case where selection of reduction subject data is to be carried out, the text display area 421 may present a message that urges selection of a specific reducing method.

The presenting screen 42 may also display an image-recapturing accepting area 423 that causes image-recapturing upon selected. For example, in a case where image-capturing is failed and the image-recapturing accepting area 423 is selected, the OCT data displayed on the OCT data selection area 422 may be deleted.

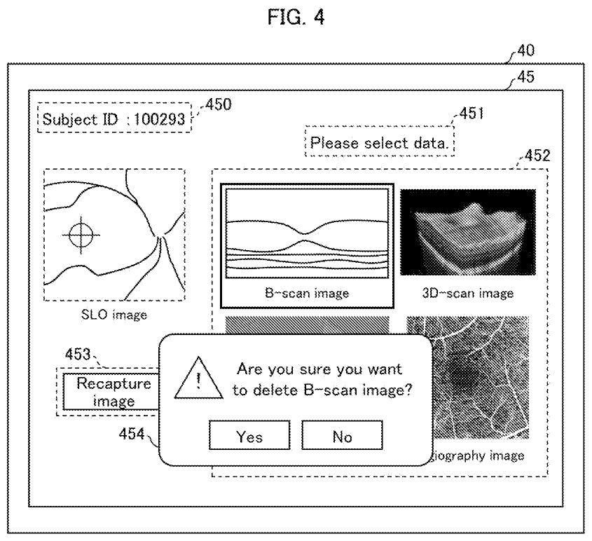

The presenting screen display section 18 may cause the display section 40 to display a presenting screen that allows confirmation of whether or not the determination of the reduction subject data or the deletion subject data made by the subject data determining section 12 is appropriate. FIG. 4 illustrates an example of a presenting screen 45 that is presented by the display section 40 and that allows confirmation of whether or not the determination of the reduction subject data or the deletion subject data is appropriate. The presenting screen 45 indicates a pop-up 454 that allows confirmation of whether or not the determination of the deletion subject data made in the presenting screen 42 shown in FIG. 3 is appropriate. For example, in a case where the B-scan image is selected as deletion subject data, a pop-up 454 indicating a message “Are you sure you want to delete B-scan image?” is displayed so as to urge confirmation of whether or not the determination of the image as the deletion subject data is appropriate. If “Yes” is selected in the pop-up 454 and the selection accepting section 15 accepts the confirmation that the image is appropriate as the deletion subject data, the reducing process section 13 carries out the deletion process. If “No” is selected in the pop-up 454 and the selection accepting section 15 accepts the confirmation that the image is not appropriate as the deletion subject data, a presenting screen that allows selection of subject data may be presented. Specifically, the presenting screen display section 18 may cause the display section 40 to display again the presenting screen that allows determination of the OCT data as non-reduction-subject data, reduction subject data, or deletion subject data.

The presenting screen that allows confirmation of whether or not the determination of the reduction subject data or the deletion subject data made by the subject data determining section 12 is appropriate is not limited to the one indicated above. Alternatively, for example, the presenting screen may indicate a list of pieces of OCT data with their respective check boxes. In such an indication, a piece(s) of data with a checked check box(es) is/are shown as non-reduction-subject data, and a piece(s) of data with a non-checked check box(es) is/are shown as deletion subject data. In this manner, a user is urged to confirm the deletion subject data.

The presenting screen display section 18 may present a presenting screen that allows selection from a plurality of templates. The template herein refers to a report in which information regarding the subject eye is blank (not written, not overlaid). For example, the template may be a report in which a piece(s) of OCT data in the above-described given image type(s), an analysis result, a subject, identification information of the subject, a finding(s), and/or the like are to be overlaid at given positions. The template may be a prescribed form or a sample of a report. The template combined with OCT data may be stored as a report in the storage section 30. Selection from the plurality of templates may be made in accordance with the subject or the subject eye. Each template includes an area(s) in which OCT data, an analysis result, and/or the like are to be overlaid, and the number and arrangement of such areas are associated in advance with their corresponding items such as OCT data, analysis results, and/or the like. In accordance with the association, a report may be created. A plurality of templates may be prepared for each kind of diseases and each lesion portion.

A plurality of templates may be prepared in advance. Each of the plurality of templates can be used to create a report including OCT data used for diagnosis of the subject. In accordance with a template allocated to the subject among these templates, the subject data determining section may determine plural pieces of OCT data as non-reduction-subject data, reduction subject data, or deletion subject data that is to be deleted. To be more specific, an image type of OCT data to be combined is determined in advance for each template. When a given template is selected, the subject data determining section 12 determines OCT data to be combined with the given template. In so doing, the subject data determining section 12 may determine, as a storage subject (non-reduction-subject data) that is to be stored without being subjected to data amount reduction, the OCT data to be combined with the given template. In addition, the subject data determining section 12 may determine the OCT data as the reduction subject data so that the OCT data is converted into a format that can be combined with the given template. Furthermore, the subject data determining section 12 may determine, as deletion subject data, OCT data that is not to be combined with the given template. With this, it is possible to store OCT data necessary for creating a report and to reduce unnecessary OCT data.

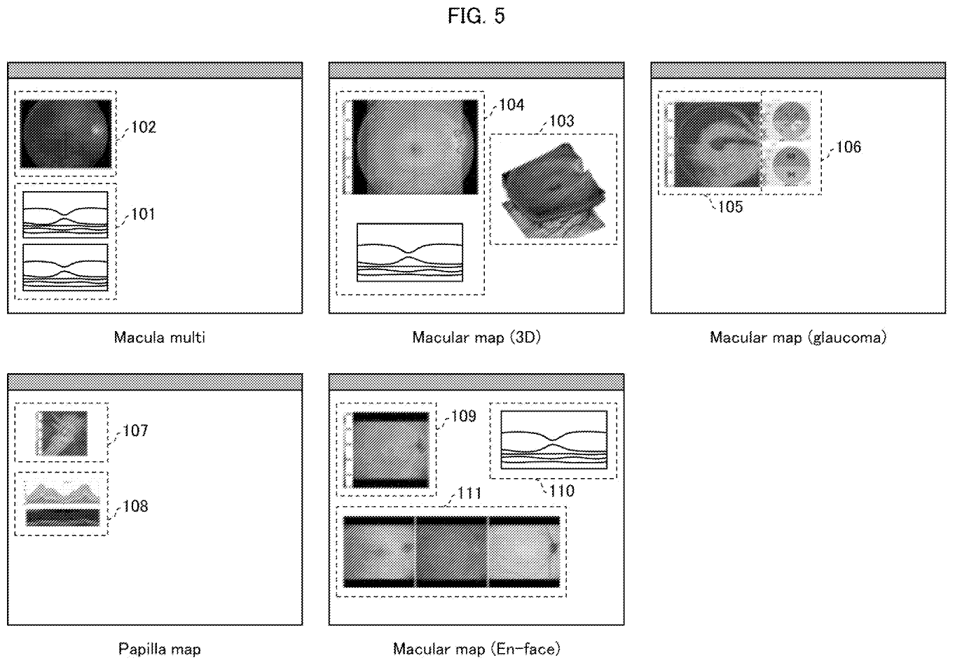

There is no particular limitation on the kinds of templates. For example, the following templates (a) to (f) may be used. If any of the templates (a) to (f) is selected, the subject data determining section 12 converts the OCT data into an image type that can be combined with the selected kind of template. FIG. 5 illustrates examples of reports prepared by combining given pieces of OCT data with the templates (a) to (f). The following description will discuss, with reference to FIG. 5, the templates (a) to (f) and the examples of the formats of OCT data to be combined with the templates.

(a) Macula Multi

A macula multi is, for example, a template to be combined with a two-dimensional tomographic image 101 showing (i) a plurality of lines in each of which a tomographic plane of a portion of a retina which portion is around a macular portion is scanned in a direction horizontal to the eyeball and (ii) a plurality of lines in each of which scanning of the eyeball is carried out in a direction perpendicular to the eyeball. There is no particular limitation on the number of lines. For example, the lines may be (i) five lines in a vertical direction and (ii) five lines in a horizontal direction. In an example, the macula multi template may also show a front image 102 of the fundus captured by another optical system.

(b) Macular Map (3D)

A macular map (3D) is, for example, a template to be combined with (i) a three-dimensional fundus image 104, which is a three-dimensional presentation of OCT data corresponding to an area around the macula, and (ii) a thickness map 103 in which different thicknesses of the macular portion are indicated in different colors.

(c) Macular Map (Glaucoma)

A macular map (glaucoma) is a template to be combined with information necessary for diagnosis of glaucoma. For example, the macular map is a template to be combined with a thickness map 105 indicating a thickness of a given layer of the retina. The given layer of the retina may be, for example, a superficial layer of the retina or all layers of the retina. The macular map (glaucoma) may be the one to be combined with an analysis chart 106 showing a result of analysis of a thickness map. The analysis chart may be, e.g., (i) a chart indicating a result of analyzing, on the basis of the thickness map, average thickness values of respective sectors in an area near the macula or (ii) a chart indicating, in a color scale, a result of comparison between the OCT data and a normative eye database.

(d) Papilla Map

A papilla map is, for example, a template to be combined with (i) a thickness map 107 indicating a thickness of a given measurement area near the papilla and (ii) an image 108 indicating a thickness distribution in each scanning line obtained in a case where circle-scanning is carried out around a center of the papilla.

(e) Macular Map (En-Face)

A macular map (En-face) is, for example, a template to be combined with (i) front images (En-face) 111 of the macular portion in various layers as viewed from the front of the eyeball and (ii) a two-dimensional tomographic image 110 of a retina tomographic plane taken along a reference line. In addition, the macular map (En-face) may be the one combined with a thickness map 109 indicating a thickness of the macular portion within a range corresponding to the front image 111.

(f) Macular Map (OCT-Angiography)

A macular map (OCT-Angiography) may be, for example, a template corresponding to a macular map (En-face) (e) in which front images (En-face) 111 of a superficial layer of the retina and a deep layer of the retina are replaced with front images of these layers obtained by OCT-Angiography.

The templates may include, in addition to the templates (a) to (f), a template to be combined with a structural abnormality degree map. The structural abnormality degree map is a map indicating a two-dimensional distribution of degrees of divergence in tissues. For example, an ophthalmologic image (OCT data) is input to a mathematical model trained by a machine learning algorithm. This gives a probability distribution used to identify the tissues in the ophthalmologic image. The degrees of divergence are degrees of divergence in the obtained possibility distribution relative to a possibility distribution with which the tissues are accurately identified, and constitute structure information indicating an abnormality degree of the tissue structure. A higher degree of divergence means a higher possibility of an abnormality in the tissue structure. There is no particular limitation on a direction of the two-dimensional distribution. In an example, the structural abnormality degree map may be a two-dimensional distribution of degrees of divergence obtained when tissues (e.g., the fundus) are viewed from the front.

The template may be the one designed for medical worker's diagnosis and/or reviewing. Alternatively, the template may be the one designed for preparation of a report to be presented in an academic conference or a report to be indicated to a third party. For example, the template may be a template suitable for preparation of a material for presentation.

Selection Accepting Section 15

The selection accepting section 15 may accept selection of non-reduction-subject data and reduction subject data in accordance with operation of the operation section 50. This will be described with reference to FIG. 3, which has been used in the description of the section “Presenting screen display section 18”. For example, the selection accepting section 15 accepts that any of the pieces of OCT data in the OCT data selection area 422 in the presenting screen 42 shown in FIG. 3 has been selected as reduction subject data. The selection may be made by, e.g., operation of the operation section 50 such as a touch panel, a controller, or the like. FIG. 3 shows the aspect in which only the deletion subject data is to be selected. Alternatively, after the deletion subject data is selected, a presenting screen that allows selection of reduction subject data or non-reduction-subject data may be presented, for example. The selection accepting section 15 may accept that any of pieces of OCT data in the presenting screen has been selected as deletion subject data.

In accordance with the selection accepted by the selection accepting section 15, the subject data determining section 12 may determine the OCT data as the non-reduction-subject data or the reduction subject data. With this, in accordance with the OCT data selected in the presenting screen 42, it is possible to store necessary OCT data and reduce unnecessary OCT data.

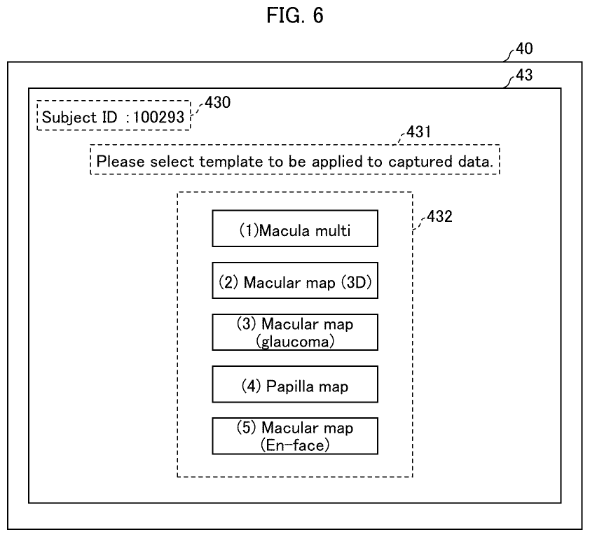

In accordance with operation of the operation section 50, the selection accepting section 15 may accept selection of a template allocated to the subject. FIG. 6 shows an example of a presenting screen 43 that allows selection from among templates presented by the display section 40. As shown in FIG. 6, the presenting screen 43 presents, for example, an identification information area 430 that displays a subject ID, a text display area 431 that displays a text, and a template selection area 432 that allows selection of a template. For example, the selection accepting section 15 accepts that any of templates, which are the options presented in the template selection area 432, has been selected. The selection may be made by, e.g., operation of the operation section 50 such as a touch panel, a controller, or the like. In accordance with the template accepted by the selection accepting section 15, the subject data determining section 12 may determine the OCT data as non-reduction-subject data or reduction subject data. With this, in accordance with the OCT data selected in the presenting screen 43, it is possible to store necessary OCT data and reduce unnecessary OCT data.

The above description has dealt with the aspect in which the template is selected after the OCT data is obtained. Alternatively, the template may be selected before the OCT data is obtained. In this case, the OCT optical system 20 may carries out image-capturing in accordance with a measurement action corresponding to the template having been selected.

Entry Accepting Section 16

The entry accepting section 16 may accept entry of an abnormal portion of the subject eye whose image has been captured by the OCT optical system. The abnormal portion is a portion arbitrarily entered by a worker in accordance with the worker's determination. For example, the abnormal portion may be, but is not limited to, a macular portion, an area around the macula, an optic nerve, and/or a blood vessel. Alternatively, the abnormal portion may be a local portion having a different luminance from that of an area around the local portion. For example, the abnormal portion may be a local portion distant from the macular portion and the papilla. A criterion for determination of an abnormality in an abnormal portion may be defined by the worker. For example, an abnormality may be determined on the basis of an abnormal value of layer thickness of each portion, a low vascular density in the superficial layer of the retina obtained by OCT-Angiography, vascular ischemia, vascular aneurysm, the presence or absence of neovascular, and/or the size of an avascular zone (FAZ).

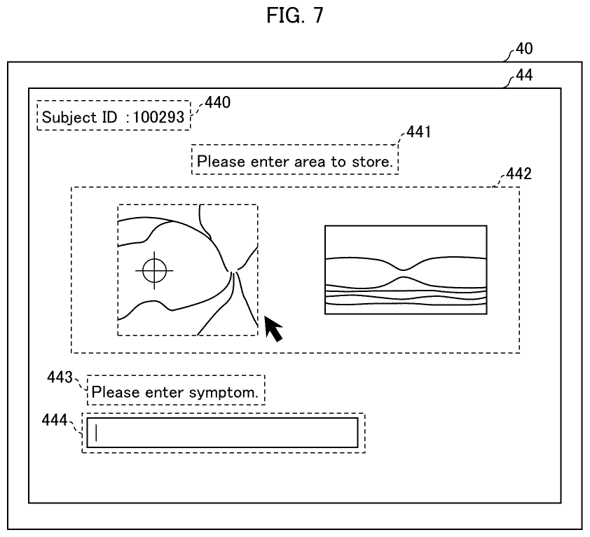

FIG. 7 illustrates an example of a presenting screen 44 that is presented by the display section 40 and that accepts entry of an abnormal portion and a symptom of the subject eye. As shown in FIG. 7, the presenting screen 44 presents, for example, an identification information area 440 that displays a subject ID, text display areas 441 and 443 that display a text, an abnormal portion entry area 442 to which an abnormal portion of the subject eye is entered, and a symptom entry area 444 to which a symptom is entered. As shown in FIG. 7, the abnormal portion entry area 442 presents, for example, an SLO image and a two-dimensional tomographic image of the subject eye. The entry accepting section 16 accepts, for example, a result of entry made by worker's arbitrary operation. Examples of the arbitrary operation encompass clicking on one point in a presented image and surrounding a certain area of the presented image with a straight line tool or a curved line tool. The operation may be made on the operation section 50, such as a touch panel or a controller. The abnormal portion entry area 442 may be an area to which a text is entered. For example, the entry accepting section 16 may accept a text entered to a text entry area, e.g., a text such as “macular portion” and/or a “papilla portion”. In accordance with the abnormal portion accepted by the entry accepting section 16, the subject data determining section 12 may determine the OCT data as non-reduction-subject data or reduction subject data. The subject data determining section 12 can determine the OCT data as the non-reduction-subject data or the reduction subject data with use of a table (not illustrated) in which abnormal portions are associated with contents and kinds of pieces of data to be stored. Specifically, in a case where the “macular portion” is entered, the subject data determining section 12 may determine the OCT data as the non-reduction-subject data or the reduction subject data with use of a table (not illustrated) in which the “macular portion” is associated with the “content and kind of data to be stored”. Further, the OCT data may be stored as a two-dimensional tomographic image of an area around the macular portion and a thickness map of the macular portion. In so doing, pieces of OCT data other than the two-dimensional tomographic image of the area around the macular portion and the thickness map of the macular portion may be deleted, for example. With this, in accordance with the abnormal portion of the subject eye, it is possible to store necessary OCT data and reduce unnecessary OCT data.

The entry accepting section 16 may accept entry of a symptom concerning the subject eye. The symptom concerning the subject eye is, for example, a given symptom observed in an abnormal portion of the above-described subject eye. The symptom concerning the subject eye may be, for example, a symptom diagnosed from an image or a symptom diagnosed as a result of a medical interview. The symptom concerning the subject eye may be arbitrarily entered by a worker. Examples of a given symptom in an area around the macula encompass (i) macular diseases such as age-related macular degeneration (AMD), a macular edema, and a macular hole and (ii) diabetic retinopathy. Examples of a given symptom in an optic nerve encompass an optic nerve disease such as glaucoma. Examples of a symptom in a local part distant from the macula and the papilla encompass detachment, a tumor, atrophy, a laser irradiation mark, ischemia, and neovascular.

The following description will discuss, with reference to FIG. 7, an aspect in which entry of a symptom is accepted. The above description has discussed, with reference to FIG. 7, the entry of the abnormal portion of the subject eye. In addition to this, the presenting screen 44 may further present the symptom entry area 444 to which a symptom is entered. The symptom entry area 444 may be, for example, an area to which a text is entered. For example, the entry accepting section 16 may accept entry of a text into the text entry area, e.g., a text such as “age-related macular degeneration” and/or “glaucoma”. In accordance with the abnormal portion entered to the abnormal portion entry area 442, the symptom entry area 444 may present options of diseases. The entry accepting section 16 may accept a result of selection made by the worker. In accordance with the symptom accepted by the entry accepting section 16, the subject data determining section 12 may determine the OCT data as non-reduction-subject data or reduction subject data. For example, the subject data determining section can determine the OCT data as the non-reduction-subject data or the reduction subject data with use of a table (not illustrated) in which symptoms are associated with contents and kinds of pieces of data to be stored. With this, in accordance with the symptom of the subject eye, it is possible to store necessary OCT data and reduce unnecessary OCT data.

A template may be selected in accordance with the abnormal portion or symptom in the subject eye accepted by the entry accepting section 16. Upon selection of the template, OCT data to be combined with the template as in the manner shown in FIG. 5 may be determined. This can be achieved by use of a table (not illustrated) in which templates are associated with necessary pieces of OCT data. With this, the template for a report corresponding to the abnormal portion or symptom in the subject eye accepted by the entry accepting section 16 is selected, and OCT data to be combined with the template is determined. With this, it is possible to store the OCT data necessary for the report corresponding to the abnormal portion or the symptom in the subject eye and to reduce unnecessary OCT data.

Subject Data Determining Method

There is no particular limitation on the method in which the subject data determining section 12 determines, on a per-piece-of-data basis, each of plural pieces of OCT data as (i) non-reduction-subject data, (ii) reduction subject data whose data amount is to be reduced more than that in a case where the each of plural pieces of OCT data is determined as non-reduction-subject data, or (iii) deletion subject data which is to be deleted. The subject data determining method may be, for example, any of the following methods.

(1) Determination in Accordance with History of Measurement on Subject

In a case where the subject from which the data obtaining section 11 obtained the plural pieces of OCT data has undergone measurement in the past, the subject data determining section 12 determines the plural pieces of OCT data as non-reduction-subject data, reduction subject data, or deletion subject data in accordance with the history of the measurement on the subject. Specifically, for example, in a case where the subject is determined, in accordance with an entered subject ID, as having undergone measurement in the past, the subject data determining section 12 can apply a previous measurement action and previous subject data also to measurement currently conducted. In a case where a criterion for a follow-up examination is defined in advance for the subject, OCT data may be determined as non-reduction-subject data, reduction subject data, or deletion subject data in accordance with the criteria. For example, assume that given OCT data serving as a reference and an analysis result obtained on the basis of the OCT data are stored for a subject with glaucoma. In this case, at the time of image-capturing for a follow-up examination, OCT data may be determined as reduction subject data or deletion subject data and only an analysis result may be determined as non-reduction-subject data. This can facilitate a follow-up eye examination for each subject.

(2) Determination on the Basis of Worker's Selection of OCT Data

Selection of non-reduction-subject data, reduction subject data, or deletion subject data may be made by a worker. In accordance with the result of the selection, the subject data determining section 12 may determine OCT data as non-reduction-subject data, reduction subject data, or deletion subject data. The selection of the OCT data may be carried out in the same manner as that described in the section “Selection accepting section 15”.

(3) Determination on the Basis of Worker's Selection of Template

In accordance with a template selected, the subject data determining section 12 may determine OCT data as non-reduction-subject data, reduction subject data, or deletion subject data. The selection of the template may be carried out in the same manner as that described in the section “Selection accepting section 15”.

(4) Determination on the Basis of Worker's Entry of Abnormal Portion of Subject Eye

In accordance with entry of an abnormal portion of the subject eye, the subject data determining section 12 may determine OCT data as non-reduction-subject data, reduction subject data, or deletion subject data. The entry of the abnormal portion may be carried out in the same manner as that described in the section “Entry accepting section 16”.

(5) Determination on the Basis of Worker's Entry of Symptom Concerning Subject Eye

In accordance with entry of a symptom concerning the subject eye, the subject data determining section 12 may determine OCT data as non-reduction-subject data, reduction subject data, or deletion subject data.

Examples of the symptom concerning the subject eye encompass the symptoms described in the section “Entry accepting section 16”. For the symptoms, the subject data determining section 12 may determine the following pieces of OCT data as non-reduction-subject data.
For a macular disease or diabetic retinopathy: A two-dimensional tomographic image obtained by multi/cross/radial scanning of an area around the macula.
For an optic nerve disease: A two-dimensional tomographic image obtained by circle-scanning around a center of the papilla, an image indicating a thickness distribution, and a thickness map of a superficial layer of the retina.
For a disease in a local portion other than the macular portion or the papilla portion: A map in which an abnormal portion is enlarged, and a two-dimensional tomographic image obtained by multi/cross/radial scanning of an area around the abnormal portion.
For vascular ischemia or vascular aneurysm: OCT-Angiography image (En-face image).
For neovascular: OCT-Angiography image (En-face image) of a layer including a lesion.

In response to entry of any of the above symptoms, a template corresponding to the symptom thus entered may be selected. The OCT data determined as non-reduction-subject data or reduction subject data may be combined with the template so as to create a report.

There may be a case where, at the time of entry of a symptom, a report including an analysis result obtained on the basis of OCT data has been already created. In such a case, the analysis result may be stored in association with the OCT data. The analysis result refers to, as described above, a result obtained by analysis of the OCT data. Examples of the analysis result encompass (i) a thickness map generated by thickness calculation from two-dimensional tomographic images and (ii) an image indicating a thickness distribution generated by thickness calculation for a certain part corresponding to a scanning line on the basis of given scanning. With this, at the time of later-conducted diagnosis, a user can view the analysis result together with the OCT data.

OCT data processing method to be executed by OCT device 1

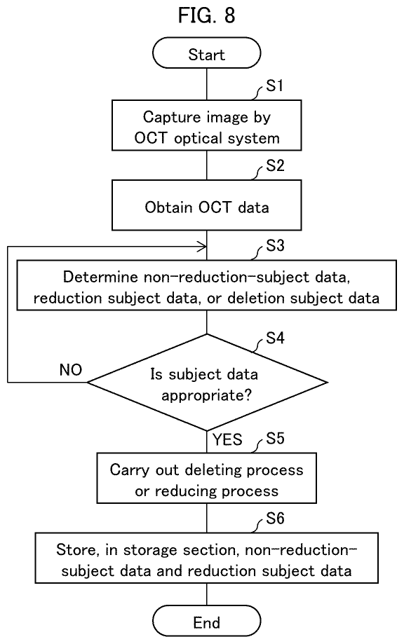

FIG. 8 is a flowchart illustrating a flow of a process to be executed by the OCT device 1 in accordance with Embodiment 1.

In S1, first, the OCT optical system 20 captures an image of the subject eye in accordance with a preset measurement action (image-capturing step).

In S2, the data obtaining section 11 obtains plural pieces of OCT data in accordance with the measurement action carried out by the OCT optical system 20 (data obtaining section).

In S3, the subject data determining section 12 determines, on a per-piece-of-data basis, each of the plural pieces of OCT data as non-reduction-subject data, reduction subject data, or deletion subject data (subject data determining step).

In S4, the presenting screen display section 18 causes the display section 40 to present a presenting screen that allows confirmation of whether or not the determination of the non-reduction-subject data, the reduction subject data, or the deletion subject data made in the subject data determining step is appropriate (subject data confirming step). If the determination of the non-reduction-subject data, the reduction subject data, or the deletion subject data is confirmed as appropriate, the process advances to S5. Meanwhile, if the determination of the non-reduction-subject data, the reduction subject data, or the deletion subject data is confirmed as not appropriate, the process returns to S3.

The above description has dealt with the aspect that allows confirmation of whether or not the determination of the non-reduction-subject data, the reduction subject data, or the deletion subject data is appropriate. However, Embodiment 1 is not limited to this. Alternatively, another aspect that allows conformation of whether or not the determination of only the reduction subject data or the deletion subject data is appropriate may be employed.

In S5, the reducing process section 13 carries out a deleting process on the deletion subject data determined by the subject data determining section 12 or a data amount reducing process on the reduction subject data determined by the subject data determining section 12 (reducing process step).

In S6, the storage executing section 14 stores, in the storage section 30, the non-reduction-subject data determined by the subject data determining section 12 and after-reduction data obtained as a result of the process carried out by the reducing process section 13 (storage executing step).

With the above procedure, the OCT device 1 can determine, on a per-piece-of-data basis, each of the obtained plural pieces of OCT data as non-reduction-subject data, reduction subject data, or deletion subject data. The reduction subject data is a subject whose data amount is to be reduced more than that in a case where the each of the obtained plural pieces of OCT data is determined as non-reduction-subject data, whereas the deletion subject data is a subject that is to be deleted. The OCT device 1 can carry out the deleting process on the deletion subject data or the data amount reducing process on the reduction subject data, and then can store the non-reduction-subject data and the after-reduction data in the storage section 30. With this, it is possible to reduce an amount of data to be stored, as compared to a case where all pieces of OCT data are stored in the storage section 30.

### Embodiment 2

The following description will discuss another embodiment of the present invention. For convenience of explanation, any member of Embodiment 2 that is identical in function to a member described for Embodiment 1 is assigned the same reference sign. Such a member is not described again here.

The description in Embodiment 1 has dealt with the aspect in which the subject data determining section 12 determines, on a per-piece-of-data basis, each of plural pieces of OCT data as a certain kind of subject data in accordance with the history of previous measurement or the result of the entry made by the worker. Specifically, the subject data determining section 12 determines, on a per-piece-of-data basis, each of the plural pieces of OCT data as (i) non-reduction-subject data, (ii) reduction subject data whose data amount is to be reduced more than that in a case where the each of the obtained plural pieces of OCT data is determined as non-reduction-subject data, or (iii) deletion subject data which is to be deleted. Meanwhile, in Embodiment 2, an OCT device 1 analyzes OCT data to detect an abnormal portion of a subject eye. The following description will discuss an embodiment in which the OCT device 1 determines, in accordance with the abnormal portion, the OCT data as (i) non-reduction-subject data, (ii) reduction subject data whose data amount is to be reduced more than that in a case where the OCT data is determined as the non-reduction-subject data, or (iii) deletion subject data which is to be deleted.

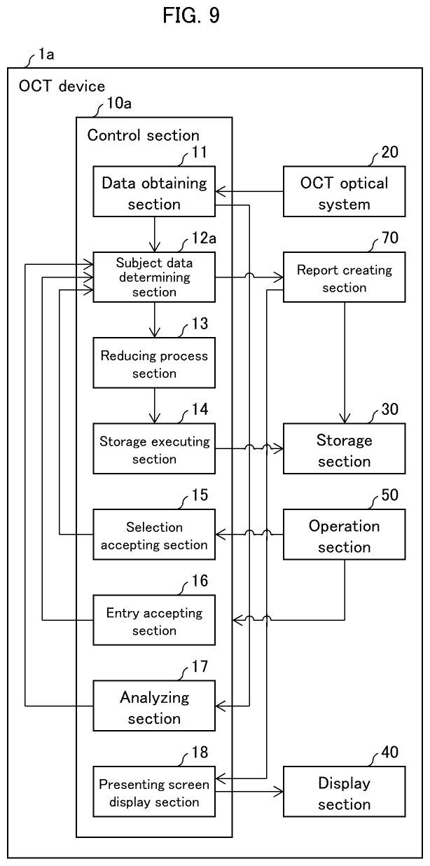

A configuration of an OCT device 1a in accordance with Embodiment 2 will be discussed with reference to FIG. 9. FIG. 9 is a block diagram illustrating an example of a configuration of a main part of the OCT device 1a in accordance with Embodiment 2 of the present invention.

The OCT device 1a include an OCT optical system 20, a control section 10a, a storage section 30, a display section 40, an operation section 50, and a report creating section 70. The control section 10a includes a data obtaining section 11, a subject data determining section 12a, a reducing process section 13, a storage executing section 14, a selection accepting section 15, an entry accepting section 16, a presenting screen display section 18, and an analyzing section 17.

Analyzing Section 17

The analyzing section 17 may analyze OCT data obtained by the data obtaining section 11 to detect an abnormal portion. The abnormal portion detected by the analyzing section 17 may be, for example, a portion corresponding to the abnormal portion described in the section “Entry accepting section 16” of Embodiment 1. The analyzing section 17 may analyze the OCT data by a known image analyzing method. In accordance with the abnormal portion detected by the analyzing section 17, the subject data determining section 12a may determine the OCT data as non-reduction-subject data, reduction subject data, or deletion subject data. With this, it is possible to automatically store OCT data corresponding to the abnormal portion of the subject eye and to reduce unnecessary OCT data.

In accordance with the abnormal portion detected by the analyzing section 17, a template corresponding to the abnormal portion may be selected. The OCT data determined as non-reduction-subject data or reduction subject data may be combined with the template so as to create a report.

The analyzing section 17 may not only detect the abnormal portion but also diagnose a disease of the subject eye. The analyzing section 17 may use, e.g., a known AI technology to diagnose the disease of the subject eye. In accordance with the disease diagnosed by the analyzing section 17, the subject data determining section 12a may determine the OCT data as non-reduction-subject data, reduction subject data, or deletion subject data. Examples of the disease to be diagnosed by the analyzing section 17 and the OCT data to be determined as the non-reduction-subject data by the subject data determining section 12a are indicated below.
In a case where the subject eye is diagnosed to have diabetic retinopathy, branch retinal vein occlusion (BRVO), or occlusion of retinal vein (CRVO): A single line extracted from a two-dimensional tomographic image, OCT-Angiography image (En-face image) of the retina.
In a case where the subject eye is diagnosed to have age-related macular degeneration or retinitis pigmentosa: A single line extracted from a two-dimensional tomographic image, OCT-Angiography image (En-face image) of choroid.
In a case where the subject eye is diagnosed to have a retinal detachment, a retinal break, or a macular hole: OCT data of the superficial layer (e.g., a superficial layer portion of a two-dimensional tomographic image, a front image of the superficial layer), and OCT data of a vitreous body.

Alternatively, in accordance with the disease diagnosed by the analyzing section 17, a template corresponding to the disease may be selected. The OCT data determined as non-reduction-subject data may be combined with the template so as to create a report.

In the embodiment in which the analyzing section 17 diagnoses a disease, the template may be the one designed for medical worker's reviewing or the one for specifying a data set required for the analyzing section 17 to carry out analysis.

### Embodiment 3

The following description will discuss another embodiment of the present invention. For convenience of explanation, any member of Embodiment 3 that is identical in function to a member described for Embodiments 1 and 2 is assigned the same reference sign. Such a member is not described again here.

An OCT device 1b in accordance with Embodiment 3 is communicable with an external device. For example, the external device may be an electric medical recording system 2 in a hospital in which the OCT device 1b is installed. In the electric medical recording system 2, reports prepared on the basis of past examinations on subjects are managed in association with pieces of personal information with which the subjects can be identified. In the electric medical recording system 2, medical workers can view a report created after an examination.

The OCT device 1b transmits a created report to the electric medical recording system 2, which is an external device, and receives a signal from the electric medical recording system 2. The electric medical recording system 2 can access OCT data stored in a storage section 30 of the OCT device 1b.

In Embodiments 1 and 2, the subject data determining section 12, 12a determines OCT data as non-reduction-subject data or reduction subject data before storing the OCT data in the storage section 30. Meanwhile, in Embodiment 3, among the pieces of OCT data stored in the storage section 30, a piece of OCT data included in the report transmitted to the external device is determined as reduction subject data.

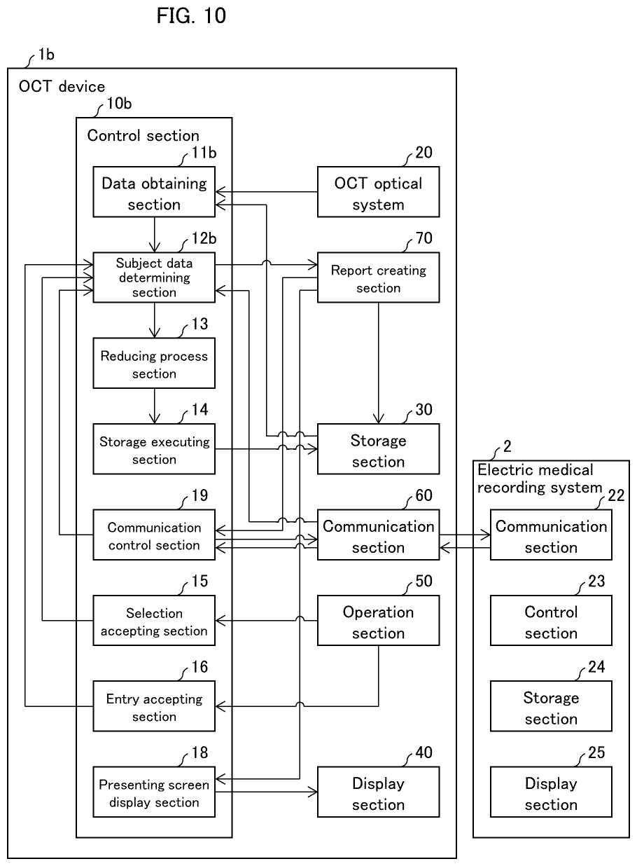

A configuration of the OCT device 1b in accordance with Embodiment 3 will be discussed with reference to FIG. 10. FIG. 10 is a block diagram illustrating an example of a configuration of a main part of the OCT device 1b in accordance with Embodiment 3 of the present invention.

The OCT device 1b include an OCT optical system 20, a control section 10b, the storage section 30, a display section 40, an operation section 50, a communication section 60, and a report creating section 70.

Communication Section 60

The communication section 60 functions as a communication device that carries out communication with an external entity under control of the later-described communication control section 19. The communication section 60 is not limited to any particular one, provided that the communication section 60 can transmit/receive a signal to/from an external entity. For example, the communication section 60 may carry out the transmission/reception by any communication method. In Embodiment 3, the communication section 60 communicates with the electric medical recording system 2, which is an external device, to transmit a report to the electric medical recording system 2, which is an external device, and to receive a signal from the electric medical recording system 2.

Control Section 10b

The control section 10b includes a data obtaining section 11b, a subject data determining section 12b, a reducing process section 13, a storage executing section 14, a selection accepting section 15, an entry accepting section 16, a presenting screen display section 18, and the communication control section 19.

Data Obtaining Section 11b

The data obtaining section 11b obtains plural pieces of OCT data from the OCT optical system 20. In addition, the data obtaining section 11b obtains, from the storage section 30, a piece of OCT data included in the report transmitted to the electric medical recording system 2 among the pieces of OCT data stored in the storage section 30.

Subject Data Determining Section 12b

The subject data determining section 12b determines the pieces of OCT data thus obtained as (i) non-reduction-subject data, (ii) reduction subject data whose data amount is to be reduced more than that in a case where the pieces of OCT data are determined as non-reduction-subject data, or (iii) deletion subject data which is to be deleted. The non-reduction-subject data is a subject that is to be stored without being subjected to data amount reduction. The subject data determining section 12b gives a flag to, among the pieces of OCT data stored in the storage section 30, the piece of OCT data included in the report already transmitted to the external device, the flag indicating that the piece of OCT data is candidate reduction subject data. The subject data determining section 12b may give the flag to (i) the piece of OCT data included in the report already transmitted to the external device among the pieces of OCT data stored in the storage section 30 or to (ii) a report that is to be transmitted. The subject data determining section 12b may retain the piece of data given with the flag in the storage section 30 in the OCT device 1b, for example, in the form of a text file. Note that the entity in which the piece of data with the flag is retained is not limited to the storage section 30 in the OCT device 1b, and is not limited to any particular one.

Communication Control Section 19

The communication control section 19 controls the communication section 60. Specifically, the communication control section 19 controls communication between the OCT device 1b and the electric medical recording system 2, which is an external device.

The communication control section 19 transmits a report created by the report creating section 70 to the electric medical recording system 2 via the communication section 60. The communication control section 19 receives a signal from the electric medical recording system 2 via the communication section 60, and outputs, to the subject data determining section 12b, an instruction corresponding to the signal thus received.

Subject Data Determining Method

The following description will discuss a method in accordance with Embodiment 3 according to which the subject data determining section 12b determines, on a per-piece-of-data basis, each of plural pieces of OCT data as non-reduction-subject data or reduction subject data.

The subject data determining section 12b may first give a flag to, among the pieces of OCT data stored in the storage section 30, a piece of OCT data included in the report already transmitted to the electric medical recording system 2, the flag indicating that the piece of OCT data is candidate reduction subject data. With this, the subject data determining section 12b can determine, in accordance with the presence or absence of the flag, whether or not the each of the plural pieces of OCT data is candidate reduction subject data.

The piece of OCT data included in the report transmitted to the electric medical recording system 2 is viewed by a medical worker on the electric medical recording system 2 side. Thus, the piece of OCT data included in the report would rarely be viewed again on the OCT device 1b side. In view of this, the subject data determining section 12b is configured to give, to the piece of OCT data included in the report having been transmitted, the flag indicating that the piece of OCT data is candidate reduction subject data. This makes it possible to smoothly carry out a subsequent OCT data reducing process on the piece of data with the flag. The subject data determining section 12b may give the flag also to a report to be transmitted to the external device. In a case where the flag is given to the report, the electric medical recording system 2 may transmit, to the OCT device 1b, a signal instructing the OCT device 1b to delete piece of OCT data that is stored in the storage section 30 and that corresponds to the piece of OCT data included in the report.

Among pieces of OCT data with a flag, pieces of OCT data corresponding to the following cases (1) to (3) may be determined as reduction subject data by the subject data determining section 12b.

(1) OCT Data Having not been Accessed by Electric Medical Recording System for Given Period

The subject data determining section 12b may determine, as reduction subject data, a piece of OCT data that has not been accessed by the electric medical recording system 2 for a given period of time after transmission of a report to the electric medical recording system 2, among pieces of OCT data given with the flag. For example, a timing at which a medical worker who has viewed a report views, on the electric medical recording system 2 side, original OCT data stored in the storage section 30 of the OCT device 1b is often immediately after an examination, e.g., immediately after transmission of the report. Further, for OCT data that a medical worker did not view on the day of the examination, the medical worker would rarely view that the OCT data another day after the examination. That is, the OCT data having not been accessed by the electric medical recording system 2 for a given period of time would be rarely viewed later. Thus, such OCT data may be determined as reduction subject data by the subject data determining section 12b. With this, the subject data determining section 12b can determine unnecessary OCT data as a reduction subject. The given period of time is not particularly limited, and may be set by a user. Specifically, the given period of time may be 12 hours or 24 hours, for example.

(2) Case where Communication Section Receives Given Signal from Electric Medical Recording System

In a case where the communication section 60 receives a given signal from the electric medical recording system 2, the subject data determining section 12 may determine a piece of OCT data with a flag as reduction subject data. Specifically, in a case where the communication section 60 in the OCT device 1b receives a given signal from a communication section 22 of the electric medical recording system 2, the subject data determining section 12 sets all pieces of OCT data with a flag as reduction subject data. That is, the signal obtained from the electric medical recording system 2 triggers the subject data determining section 12 determining a piece of OCT data with a flag as reduction subject data. The given signal will be described below.

(2-1) Response Signal Indicating that Electric Medical Recording System has Received Report

When the communication section 60 receives a response signal indicating that the electric medical recording system 2 has received a report, the subject data determining section 12b may determine a piece of OCT data with a flag as reduction subject data. That is, a piece of OCT data included in a report transmitted from the OCT device 1b to the electric medical recording system 2 without any trouble is determined as a reduction subject, since such a piece of OCT data, which is original data, is rarely viewed again later.

(2-2) Signal Indicating that Report is Opened in Electric Medical Recording System

When the communication section 60 receives a signal indicating that a report received from the OCT device 1b is opened in the electric medical recording system 2, the subject data determining section 12b may determine a piece of OCT data with a flag as reduction subject data. That is, when the report is opened, i.e., the report is viewed in the electric medical recording system 2, the piece of OCT data included in the report is determined as a reduction subject.

(3) Case where Report Created Based on n+2-th Examination on Subject is Transmitted to Electric Medical Recording System

A piece of OCT data obtained in an examination on a subject, conducted m times (m≥1) before a previous examination which has been conducted on the same subject and as a result of which a piece of OCT data given with a flag has been obtained, may be determined as a reduction subject. In a case where a report including a piece of OCT data obtained in an n+m-th examination, conducted on a subject having undergone a previous examination as a result of which a piece of OCT data given with a flag has been obtained, is transmitted to the electric medical recording system 2, the subject data determining section 12b may determine, as reduction subject data, pieces of OCT data obtained in the first to n-th examinations. A value of m may be set as appropriate. A medical worker, i.e., a user, may set the value of m in advance. The value of m may be set for each subject.

For a subject who has undergone an examination a plurality of times, it is not necessary to retain all pieces of OCT data obtained in the past examinations. For example, as long as pieces of OCT data obtained in latest two examinations are remained, a doctor can carry out diagnosis without any trouble in most cases. In this case, the value of m is set at 2. Specifically, in a case where a report including a piece of OCT data obtained in the third examination is transmitted to the electric medical recording system 2, the subject data determining section 12b may determine, as reduction subject data, a piece of OCT data obtained in the examination (the first examination) before the last examination. In this case, the first examination may be the examination conducted at the subject's first consultation.

As described above, a single examination may be examinations carried out on a single subject on a single day. Thus, a piece of OCT data obtained in an examination on a subject, conducted m days (m≥1) before a previous examination which has been conducted on the same subject and as a result of which a piece of OCT data given with a flag has been obtained, may be determined as a reduction subject. In a case where a report including a piece of OCT data obtained in an examination on an n+m-th day, conducted on a subject having undergone a previous examination as a result of which a piece of OCT data given with a flag was obtained, is transmitted to the electric medical recording system 2, the subject data determining section 12b may set, as reduction subject data, pieces of OCT data obtained on the first to n-th days.

### Embodiment 4

In Embodiment 3, the subject data determining section 12b determines, as candidate reduction subject data, the piece of OCT data included in the report transmitted to the external device, among the pieces of OCT data stored in the storage section 30. Then, the subject data determining section 12b determines reduction subject data from among the candidate data. Similarly to Embodiment 3, an OCT device 1b in accordance with Embodiment 4 can communicate with an external device one another. However, a subject data determining section 12b in the OCT device 1b in accordance with Embodiment 4 determines subject data in a different manner from that of Embodiment 3.

Subject Data Determining Method

An OCT image included in a report transmitted to the electric medical recording system 2 is used by a doctor to carry out diagnosis, and thus can be considered as an important OCT image. In view of this, the subject data determining section 12b may determine, as non-reduction-subject data, a piece of OCT data included in the report, and may determine, as reduction subject data, pieces of OCT data other than the piece of OCT data included in the report. With this, for example, when the doctor wishes to view the piece of OCT data included in the report, the doctor can view the piece of OCT data stored in the storage section 30.

The subject data determining section 12b may determine, as non-reduction-subject data, a piece of OCT data included in a report and pieces of OCT data obtained consecutively to the piece of OCT data included in the report, and may determine, as reduction subject data, pieces of OCT data other than those pieces of OCT data. The pieces of OCT data obtained consecutively to the piece of OCT data refer to, among plural pieces of OCT data obtained by image-capturing carried out a plurality of times at slightly shifted scanning positions, some consecutive slice images preceding or following the piece of OCT data of question. Needless to say, the piece of OCT data included in the report includes information useful for diagnosis. A piece of OCT data consecutive to the piece of OCT data included in the report may also possibly include useful information. Therefore, the piece of OCT data included in the report and the pieces of OCT data obtained consecutively to the piece of OCT data included in the report are determined as non-reduction-subject data, and the pieces of OCT data other than those pieces of OCT data are determined as reduction subject data. With this, it is possible to retain necessary OCT data and reduce unnecessary OCT data.

The subject data determining section 12b may determine, as non-reduction-subject data, a piece of OCT data including information indicating an area having an anatomical characteristic among plural pieces of OCT data. The area having an anatomical characteristic may be, for example, fovea or optic papilla. A piece of OCT data including information indicating an area other than the area having an anatomical characteristic may be determined as reduction subject data. With this, it is possible to retain only OCT data including information indicating an area having an anatomical characteristic, which will highly probably be viewed again, and to reduce unnecessary OCT data which will rarely be viewed again.

In a case where a report including the above-described structural abnormality degree map is transmitted to the electric medical recording system 2, the subject data determining section 12b may determine, as non-reduction-subject data, OCT data exhibiting a higher degree of divergence than a given value in the structural abnormality degree map. The OCT data having a higher degree of divergence than the given value is OCT data corresponding to a tissue structure with an abnormality. Meanwhile, OCT data having a lower degree of divergence than the given value, i.e., OCT data corresponding to a tissue structure without an abnormality may be determined as reduction subject data. With this, it is possible to retain only OCT data corresponding to a tissue structure with an abnormality, which will highly probably be viewed again.

In order to create various analysis maps such as macula multi and/or papilla map, plural pieces of OCT data are used. Some of the pieces of OCT data that are obtained from image-capturing and that are used to create different kinds of analysis maps have the same contents. The subject data determining section 12 may determine, as non-reduction-subject data, only one of such pieces of OCT data having the same contents, and may determine the others as reduction subject data. For example, in a case where pieces of OCT data used to create a macular line are included in pieces of OCT data used to create a macular map, the subject data determining section 12 may determine, as a reduction subject, these pieces of OCT data included in the macular line.

A piece of OCT data included in a report transmitted to the electric medical recording system 2, which is an external device, may be stored in a highly compressed state. There is no particular limitation on a storage form. In an example, before transmission of a report to the electric medical recording system 2, OCT data may be stored in a bitmap format. After transmission of the report, the OCT data may be converted into a highly-compressed image format, such as JPEG, so as to be stored in the storage section 30.

Variations

The following description will discuss variations of the present invention. Any of the variations may be applied to any of the above-described embodiments. For convenience of explanation, any member of each variation that is identical in function to a member described for the above-described embodiments is assigned the same reference sign. Such a member is not described again here.

A variation of the reducing process to be carried out by the reducing process section 13 will be described.
The reducing process section 13 may not retain a two-dimensional tomographic image, and may extract only a line representing a layer boundary obtained by layer-boundary detection and delete the other portions.
The reducing process section 13 may decimate, among pieces of obtained OCT data, a piece of OCT data indicating information in a layer thickness direction or pieces of OCT data indicating information in all directions. That is, the reducing process section 13 may delete a piece(s) of OCT data other than a piece(s) of OCT data at a given distance(s) in the thickness direction.
In a case where the analyzing section 17 carries out detection of an abnormal portion, only an area corresponding to the abnormal portion indicated by the OCT data may be obtained at a high resolution. The reducing process section 13 may reduce a resolution of an area other than the abnormal portion.
For each subject, the reducing process section 13 may store all pieces of OCT data obtained by image-capturing carried out at his/her first consultation. Then, only for a subject who needs a continued follow-up examination (follow-up), the reducing process may be applied at his/her second and subsequent medical consultations.
The contents of the reducing process and the deleting process to be carried out by the reducing process section 13 may be set in advance for each subject or for each medical facility, for example. Switching may be allowed between a mode of carrying out the reducing process and a mode of not carrying out the deleting process.

In Embodiment 3, the reducing process section 13 reduces the OCT data included in the report that has been transmitted to the electric medical recording system 2 and that has been determined as a reduction subject by the subject data determining section 12b. Here, the reduction subject data may be archived in a storage other than the electric medical recording system 2, e.g., in a cloud storage. The archiving means transferring the OCT data into another storage device, a recording medium, or the like each available at a lower cost. The archiving may be carried out by the communication control section 19.

The reducing process section 13 may process the reduction subject data without carrying out the reducing process on the reduction subject data. The processing may be, e.g., an anonymous process that makes the subject of the reduction subject data unidentifiable. The data having been processed to make the subject unidentifiable in this manner may be transferred via the communication control section 19 so as to be stored in, e.g., a cloud storage that is not the electric medical recording system 2. The data having been processed may be used as data for machine learning.

### Software Implementation Example

The features of the OCT device 1 (hereinafter, referred to as a “device”) can be realized by a program for causing a computer to function as the device, the program causing the computer to function as each control block (particularly, each of the sections included in the control section 10) of the device.

In this case, the device includes, as hardware for executing the program, a computer including at least one control device (e.g., a processor) and at least one storage device (e.g., a memory). By causing the control device and the storage device to execute the program, it is possible to achieve the features described in the above-described embodiments.

The program may be stored in one or more non-transitory, computer-readable recording media. The one or more recording media may or may not be included in the device. In the latter case, the program may be supplied to the device via any transmission medium connected by wired or wireless communication.

All or part of the features of the above-described control blocks can be realized by a logic circuit. For example, an integrated circuit in which the logic circuit functioning as each of the control blocks is also encompassed in the scope of the present invention. Alternatively, for example, the features of the control blocks can be realized by a quantum computer.

The processes described in the above-described embodiments may be executed by artificial intelligence (AI). In this case, AI may be operated in the control device or in another device (e.g., an edge computer or a cloud server).

The present invention is not limited to the embodiments, but can be altered by a skilled person in the art within the scope of the claims. The present invention also encompasses, in its technical scope, any embodiment derived by combining technical means disclosed in differing embodiments.

### REFERENCE SIGNS LIST

1: OCT device
10, 10a, 10b: control section
11: data obtaining section
12: subject data determining section
13: reducing process section
14: storage executing section
15: selection accepting section
16: entry accepting section
17: analyzing section
18: presenting screen display section
20: OCT optical system
30: storage section
40: display section
41, 42, 43: presenting screen
60: communication section

1. An OCT device comprising:
an OCT optical system that captures an image of a subject eye of a subject in accordance with a measurement action;
a data obtaining section that obtains plural pieces of OCT data in accordance with the measurement action;
a subject data determining section that determines, on a per-piece-of-data basis, each of the plural pieces of OCT data as (i) non-reduction-subject data or (ii) reduction subject data whose data amount is to be reduced more than that in a case where the each of the plural pieces of OCT data is determined as the non-reduction-subject data or which is to be deleted;
a reducing process section that carries out a data amount reducing process or a deleting process on the reduction subject data determined by the subject data determining section; and
a storage executing section that stores, in a storage section, the non-reduction-subject data determined by the subject data determining section and after-reduction data obtained as a result of the data amount reducing process carried out on the reduction subject data by the reducing process section.

2. The OCT device as set forth in claim 1, wherein
the data obtaining section obtains a given piece of OCT data, which is a piece of OCT data obtained at a preset scanning position in the subject eye of the subject, and
the storage executing section stores, in the storage section, the given piece of OCT data in addition to the non-reduction-subject data and the after-reduction data.

3. The OCT device as set forth in claim 1, wherein
the plural pieces of OCT data are obtained in a single examination carried out on the subject, the examination including the measurement action, and
the subject data determining section determines, as non-reduction-subject data or reduction subject data, each of the plural pieces of OCT data obtained in the examination, the determination being carried out on a per-piece-of-data basis.

4. The OCT device as set forth in claim 1, wherein
a plurality of templates are prepared in advance, each of the plurality of templates being for creating a report including at least one of the plural pieces of OCT data, and
the subject data determining section determines, on a per-piece-of-data basis, each of the plural pieces of OCT data as non-reduction-subject data or reduction subject data in accordance with a template allocated to the subject among the plurality of templates.

5. The OCT device as set forth in claim 4, further comprising:
a presenting screen display section that causes a display section to display a presenting screen that allows selection of the template; and
a selection accepting section that accepts the selection of the template allocated to the subject, wherein
the subject data determining section determines, on a per-piece-of-data basis, each of the plural pieces of OCT data as non-reduction-subject data or reduction subject data in accordance with the template selected in the presenting screen.

6. The OCT device as set forth in claim 5, wherein
in a case where the report includes an analysis result of the at least one of the plural pieces of OCT data, the storage executing section stores, in the storage section, the analysis result of the at least one of the plural pieces of OCT data in association with the non-reduction-subject data.

7. (canceled)

8. The OCT device as set forth in claim 4, further comprising:
a communication section that transmits the report to an external device and receives a signal from the external device, wherein
the subject data determining section gives a flag to, among the pieces of OCT data stored in the storage section, the at least one of the plural pieces of OCT data included in the report transmitted to the external device, the flag indicating that the at least one of the plural pieces of OCT data is candidate reduction subject data.

9. The OCT device as set forth in claim 8, wherein
the external device is accessible to the plural pieces of OCT data stored in the storage section, and
the subject data determining section determines, as reduction subject data, a piece of OCT data that has not been accessed by the external device for a given period of time after transmission of the report to the external device, among the at least one of the plural pieces of OCT data given with the flag.

10. The OCT device as set forth in claim 8, wherein
in a case where the communication section receives a given signal from the external device, the subject data determining section determines, as reduction subject data, the at least one of the plural pieces of OCT data given with the flag.

11. (canceled)

12. (canceled)

13. The OCT device as set forth in claim 8, wherein
in a case where a report including a piece of OCT data obtained in an n+m-th (m≥1) examination on the subject who has undergone a previous examination as a result of which the at least one of the plural pieces of OCT data given with the flag has been obtained is transmitted to the external device, the subject data determining section sets, as reduction subject data, pieces of OCT data obtained in first to n-th examinations, among the at least one of the plural pieces of OCT data given with the flag.

14. (canceled)

15. The OCT device as set forth in claim 1, further comprising:
a presenting screen display section that causes a display section to present a presenting screen that allows confirmation of whether or not the determination of the reduction subject data made by the subject data determining section is appropriate; and
a selection accepting section that accepts a result of the confirmation of whether or not the determination of the reduction subject data is appropriate, wherein
the presenting screen includes an image-recapturing accepting area that causes image-recapturing upon selected.

16. The OCT device as set forth in claim 1, further comprising:
an entry accepting section that accepts entry of an abnormal portion of the subject eye whose image has been captured by the OCT optical system, wherein
the subject data determining section determines, on a per-piece-of-data basis, each of the plural pieces of OCT data as non-reduction-subject data or reduction subject data in accordance with the abnormal portion accepted by the entry accepting section.

17. The OCT device as set forth in claim 1, further comprising:
an entry accepting section that accepts entry of a symptom concerning the subject eye, wherein
the subject data determining section determines, on a per-piece-of-data basis, each of the plural pieces of OCT data as non-reduction-subject data or reduction subject data in accordance with the symptom accepted by the entry accepting section.

18. The OCT device as set forth in claim 1, further comprising:
an analyzing section that analyzes the plural pieces of OCT data obtained by the data obtaining section to detect an abnormal portion of the subject eye, wherein
the subject data determining section determines, on a per-piece-of-data basis, each of the plural pieces of OCT data as non-reduction-subject data or reduction subject data in accordance with the abnormal portion detected by the analyzing section.

19. The OCT device as set forth in claim 1, wherein
each of the plural pieces of OCT data obtained by the data obtaining section is at least one of (i) two-dimensional OCT data indicating two-dimensional information extending in a direction intersecting an optical axis of the OCT optical system and (ii) three-dimensional OCT data indicating three-dimensional information extending in a direction intersecting the optical axis and a direction along the optical axis.

20. The OCT device as set forth in claim 1, wherein
in a case where the subject from which the data obtaining section obtained the plural pieces of OCT data has a history that the subject has undergone measurement in past, the subject data determining section determines, on a per-piece-of-data basis, each of the plural pieces of OCT data as non-reduction-subject data or reduction subject data in accordance with the history of the subject.

21. The OCT device as set forth in claim 1, wherein
each of the plural pieces of OCT data includes information of at least one of a macular portion, an optic papilla portion, and vasa sanguinea retinae of the subject eye.

22. A method for processing OCT data, comprising:
an image-capturing step of capturing an image of a subject eye of a subject in accordance with a measurement action;
a data obtaining step of obtaining plural pieces of OCT data in accordance with the measurement action;
a subject data determining step of determining, on a per-piece-of-data basis, each of the plural pieces of OCT data as (i) non-reduction-subject data or (ii) reduction subject data whose data amount is to be reduced more than that in a case where the each of the plural pieces of OCT data is determined as the non-reduction-subject data or which is to be deleted;
a subject data confirming step of allowing confirmation of whether or not the determination of the reduction subject data made in the subject data determining step is appropriate;
a reducing process step of carrying out a deleting process or a data amount reducing process on the reduction subject data determined in the subject data determining step; and
a storage executing step of storing the non-reduction-subject data determined in the subject data determining step and after-reduction data that is obtained as a result of the data amount reducing process carried out on the reduction subject data in the reducing process step.

23. A program for causing a computer to function as the OCT device recited in claim 1, the program causing the computer to function as the subject data determining section, the reducing process section, and the storage executing section.

24. A computer-readable recording medium in which the program recited in claim 23 is stored.
